(19) United States
(12) Patent Application Publication
(10) Pub. No.: US 2009/0311351 A1
(43) Pub. Date: Dec. 17, 2009

(54) COMPOSITION CONTAINING A CEREAL MILLING PRODUCT

(75) Inventor: Johannes F. COY, Otzberg (DE)

Correspondence Address: MORRISON & FOERSTER LLP, 12531 HIGH BLUFF DRIVE, SUITE 100, SAN DIEGO, CA, 92130-2040, US

(73) Assignees: Johannes F. Coy, Otzberg (DE); Mestemacher GmbH, Gutersloh (DE)

(21) Appl. No.: 12/479,539

(22) Filed: Jun. 5, 2009

Related U.S. Application Data
(63) Continuation of application No. PCT/EP2007/010660, filed on Dec. 7, 2007.

(30) Foreign Application Priority Data
Dec. 7, 2006 (DE) 20 2006 018 834.7

(51) Int. Cl. A61K 36/899; A61K 36/55; A61P 35/00; A61P 3/10; A61P 25/00; A61P 25/28

(52) U.S. Cl. 424/750; 424/768

(57) ABSTRACT

The invention relates to a composition, in particular a bread, containing a cereal milling product, wherein the composition has a carbohydrate fraction of less than 15% by weight and an omega-3 fatty acid content of at least 2% by weight.

### CROSS-REFERENCE TO RELATED APPLICATIONS

This application is a continuation of international patent application PCT/EP2007/010660 filed on Dec. 7, 2007, and designating the U.S., which international patent application has been published in German language and claims priority from German utility patent DE 20 2006 018 834.7 filed on Dec. 7, 2006. The entire contents of these priority applications are incorporated herein by reference.

### BACKGROUND OF THE INVENTION

The present invention relates to a composition containing a cereal milling product which has a defined content of carbohydrates, fats and proteins.

Such compositions are generally processed to or represent in particular baked goods, such as, for example, bread or rolls, or added to bread mixes or the like. Bread is a basic food made from cereals in milled or coarse-meal form, water, salt and raising agents, and can have further components such as fat, sugar, milk products, potato products, spices.

Foods containing cereal milling products, such as bread, for example, supply not only energy, but also valuable nutrients and dietary fibres. For healthy nutrition, the exact composition of such foods is of outstanding importance, since many baked goods, that is also many breads, owing to their components do not conform to the demand of healthy and balanced nutrition. In particular for people who, for example, owing to metabolic diseases/disorders, must pay attention to their nutrition, or who simply want to eat consciously, the exact composition of foods containing cereal products plays an important role in the daily nutrition plan.

For instance diabetes mellitus patients, for example, must very particularly pay attention to their blood sugar level, since a poorly set blood sugar level leads to secondary diseases such as, for example, damage to the retina, or damage to the kidneys.

Diabetes has become a mass disease, in particular in the industrial nations, with the main causes being overeating, lack of movement and the resulting overweight. An excessive blood sugar level leads to chronic long-term damage such as, for example, retinal disease, and also to nerve damage and blood vessel damage.

However, for other diseases such as neurodegenerative diseases, such as Alzheimer's disease, or cancer, it has also recently been found that targeted and conscious nutrition plays an important role. For these diseases also, it has been shown that their symptoms can be influenced for the better in a targeted manner by a conscious selection of the composition of daily foods.

Since in particular foods such as bread or rolls, for example, are basic foods and are therefore consumed in some form usually daily, the compositions and components of such foods play a great role in nutrition.

As mentioned above, the compositions of, e.g., bread generally have relatively large fraction of milled cereals, in addition water, a raising agent and also if appropriate further components such as, for example, sunflower seeds or pumpkin seeds, fruits, milk products, etc. In particular, the nutritional value of bread and also its fat and dietary fibre fractions can be influenced by selection of the cereal.

The (bread) compositions known in the prior art have the disadvantage that they are predominantly unsuitable for people who, on account of, for example, metabolic disorders, are instructed to eat a very defined composition, and rather, their consumption could further worsen the disease profile. For instance, conventional breads, for example, are usually unsuitable as a daily basic food for diabetics, since consumption of carbohydrate-rich baked goods increases the blood sugar level extremely.

In addition, the components of the breads or bread varieties known in the prior art are frequently only harmonized with respect to good flavour, but not to defined nutritional values which, even in the case of healthy people, can make a significant contribution to general well being.

It is therefore an object of the present invention to provide a composition containing a cereal milling product, in particular a bread composition, which can be consumed even by people having disordered metabolism, which further can even be health-promoting for people having certain diseases and which makes a health contribution to the human organism generally.

### SUMMARY OF THE INVENTION

According to the invention, this object is achieved by providing a composition containing a cereal milling product which has a carbohydrate fraction of less than 15% by weight and an omega-3 fatty acid content of at least 2% by weight, and preferably in addition has a fat content of at least 10% by weight and a protein content of at least 12% by weight.

The object is further achieved by a baked good which has the composition of the invention.

The object of the invention is completely achieved in this manner.

### DETAILED DESCRIPTION OF THE INVENTION

The composition of the invention, e.g., a bread, ensures, inter alia, that on account of the low carbohydrate fraction, only little glucose is released, such that, for example, a bread which has the composition of the invention can also be eaten by diabetics. In addition, the body, owing to the consumption of the composition of the invention, is simultaneously supplied with essential fats, as a result of which the base supply with these vital nutrients is ensured.

The composition of the invention, for example a bread, has, compared with the breads known in the prior art, significantly less carbohydrates: depending on the cereal variety, the carbohydrate fraction of a “standard” bread can vary between approximately 35% by weight and 50% by weight. On consumption of such breads, therefore large amounts of glucose are released which can lead to chronic long-term damage. By means of the composition of the invention which has a carbohydrate fraction of less than approximately 15% by weight, the high glucose release is advantageously avoided.

In particular, taking into account relatively recent studies which have found that glucose is used as sole fuel in certain types of cancer, and also plays a role in neurodegenerative diseases (see, for example, Coy, et al., Clin. Lab. (2005) 51:257-273), by means of the composition of the invention, a simple health-promoting nutritional basis can be provided in these diseases. The composition of the invention thus offers the possibility of controlling the glucose level in a targeted manner instead of using, or in combination with, drugs, as a result of which the course of the disease and simultaneously the quality of life can be improved.

In addition, the composition of the invention, again in contrast to the cereal-containing food compositions known in the prior art, has a very high content of omega-3 fatty acids.

Omega-3 fatty acids are essential for humans and are not formed by the body itself, which is why they must be consumed with the food. Relatively recent studies have demonstrated the health-promoting action of omega-3 fatty acid such as, for example, their antithrombotic action which can prevent cardiovascular disorders. In addition, omega-3 fatty acids are required for development of the brain and vision. It has also been found that an omega-3-fatty acid-rich diet is health-promoting for humans having rheumatic disorders, because hormone-like substances formed from them develop an anti-inflammatory action.

With the composition of the invention, therefore a health-promoting action is achieved which cannot be achieved with the cereal-containing food compositions known in the prior art.

According to one aspect, the omega-3 fatty acid content is provided by shelled hemp seeds, linseed, sesame, walnuts, rapeseed or soya.

Of these foods it is known that they have a high omega-3 fatty acid content which is why they are suitable as components for the composition of the invention, either in each case alone or else in various combinations. The choice of the respective omega-3 fatty acid donor is made in each case dependent on which target group the composition, for example in bread form, is intended. Obviously, for example in the case of people with allergies, such as, for example nut allergies, the use of walnuts will be avoided.

In addition, and according to another aspect, other additional components which are conventional in breads or bread-like foods can be used in the composition of the invention such as, for example, dietary fibres, water, flavourings, fruits, vitamins, salt etc. It will be clear to those skilled in the art that according to which target group/for which disorders the composition is to be used, many different components or supplements can be added in different amounts and combined with one another, with at the same time the percentage fraction according to the invention of carbohydrates and omega-3 fatty acids essentially being maintained in the composition.

According to another aspect, the composition of the invention comprises the following: 25 to 35% by weight cereal milling products, 7 to 17% by weight hemp seeds, 7 to 17% by weight soya meal, 1 to 5% by weight linseed, 0.5 to 4.5% by weight protein and 0.5% to 2% sesame seed.

According to one aspect, the cereal milling product is based on rye or spelt or on a cereal type whose amylopectin fraction is significantly greater than its amylose fraction.

Rye contains much iron and is rich in dietary fibre. Spelt is exceedingly nutritious and readily digestible. Both types of cereal have a higher dietary fibre fraction and a lower gluten fraction than wheat and are also particularly suitable for this reason for the composition of the invention. The starch present in the cereal varieties is made up of amylose and amylopectin, with glucose in the amylose being linked via alpha-1,4-bonds, and in the amylopectin additionally via alpha-1,6-branches. On account of the additional 1,6-branches of amylopectin, this is broken down more slowly by amylase into its basic units, that is glucose, which in turn lowers the total content of glucose.

According to one aspect, the composition or the bread of the invention comprises the following:
approximately 29% by weight rye milling products,
approximately 12% by weight hemp seeds
approximately 12% by weight soya meal,
approximately 3% by weight linseed,
approximately 2.5% by weight protein,
approximately 1.5% by weight sesame seed
approximately 1.5% by weight wheat bran
approximately 1.5% by weight milk protein, and also
approximately 1% by weight rice flakes.

This embodiment of the composition or the bread according to the invention has proved particularly suitable in experiments, not only with respect to health but also with respect to the production method of baked goods which comprise or represent the composition according to the invention.

The abovementioned ingredients are provided in accordance with their final fraction in the finished composition or the finished bread and mixed with addition of a liquid such as water and, in the case of a baked good, baked.

The invention further relates to a method for treating or preventing a disease in a human in need thereof, wherein the disease is selected from the group consisting of cancer types which have an active TKTL-1 metabolism, diabetes, and neurodegenerative diseases, in particular Alzheimer's disease, comprising the step of administering to said human a therapeutically effective amount of the composition according to the invention.

The inventor of the present composition has found that certain aggressive cancer cells which ferment glucose instead of burning it cover their energy requirements only with glucose and can utilize neither fat nor oils as a further energy source. An important part in this altered metabolism in tumours is played by the enzyme transketolase-like 1 (TKTL 1) which is strongly expressed in aggressive cancer cells and is correspondingly active. By means of the composition according to the invention, now an effective and promising means is provided which can be used for therapy of cancer types in which TKTL 1 is up-regulated and in which glucose fermentation is active. Owing to the low carbohydrate fraction in the composition according to the invention, this is because low glucose release is ensured. As a result, in the tumours and in the cancer cells little glucose is fermented and therefore the activity of TKTL 1 is decreased, as a result of which, again the growth of the tumours can be braked. Therefore, the composition according to the invention, in particular owing to the combination of a low carbohydrate fraction and a high omega-3 fatty acid fraction, is an excellent means for counteracting tumour diseases, for example even via feeding with basic foods such as bread.

As already mentioned above, the composition according to the invention is also suitable in the therapy of diabetes mellitus, since it ensures that only very little glucose is released which lowers the, otherwise dangerously high, glucose level in the blood of diabetes patients. In addition, the simultaneous administration of vitamin B1 or vitamin B1 derivatives (for example benfotiamin) can be provided in the composition which gives a cofactor for transketalase enzymes. As a result the TKTL 1 enzyme is activated, as a result of which in turn three main metabolic pathways which lead to hyperglycemic damage are blocked. Thus the formation of Advanced Glycation End products (AGEs) is decreased, that is products which are formed in patients with diabetes mellitus by glycosylation of, inter alia, proteins. In addition, the activation of the TKTL 1 enzyme reduces the formation of ROS (reactive oxygen species) which leads to decreased cell damage and cell ageing (“antiageing”).

In the case of neurodegenerative disorders such as Alzheimer's disease, the composition of the invention can also be used as means for therapy or prevention. For instance it has been found, for example in the case of Alzheimer's disease, that glucose metabolism and the formation of AGEs play a role for this disease and for ageing itself, since AGEs are present, for example in senile plaques which are found in great density in the grey matter of the brains of Alzheimer's patients. The low-carbohydrate composition can therefore achieve an advantageous lowering of the glucose level in these patients, such that the formation of AGEs and their deposition in plaques can also be prevented.

The composition can be administered in form of baked goods, in particular bread, wherein the baked goods, in particular the bread, can be administered daily. Depending on the human to be treated, i.e., his or her overall condition, age, weight, etc., the amount or dose of the composition/bread may vary, and it lies within the knowledge and ability of the treating person skilled in the art, to determine the effective amount, respectively.

An embodiment of the invention is described in more detail in the example hereinafter.

### EXAMPLE

For the food composition, here: a bread, the following ingredients were prepared: for the base of the cereal milling product, rye was chosen, in addition hemp seed, soya meal, linseed, sesame seed, wheat bran and rice flakes. The ingredients were mixed with water and baked at 210° C. for 70 min to give bread.

Subsequently the components were determined according to their fraction. In Table 1 below, the nutritional value analysis of the baked good is represented:

TABLE 1
Nutritional value analysis

| | | |
|---|---|---|
| Calorific value | /100 g | 832(199) |
| Protein | g/100 g | 12.2 |
| Carbohydrates | g/100 g | 14.1 |
| Fat | g/100 g | 10.4 |
| Dietary fibre | g/100 g | 14.2 |
| Water | /100 g | 46.8 |
| Ash | g/100 g | 2.3 |
| Bread units | /100 g | 1.2 |
| Equivalent to 1 BU | g | 85 |
| Omega-3 fatty acids | % by weight | 2.2 |

As follows from the example, the finished bread contains less than 15% by weight carbohydrates and more than 2% by weight omega-3 fatty acids and is therefore suitable in the context of a dietetic nutritional plan for example also for diabetics.

1. A composition containing a cereal milling product, the composition having a carbohydrate fraction of less than 15% by weight and an omega-3 fatty acid content of at least 2% by weight, wherein the composition further contains:
25 to 35% by weight cereal milling products,
7 to 17% by weight hemp seeds,
7 to 17% by weight soya meal,
1 to 5% by weight linseed,
0.5 to 4.5% by weight protein, and
0.5 to 2% sesame seed.

2. The composition of claim 1, wherein in addition it has a fat content of at least 10% by weight and also a protein content of at least 12% by weight.

3. The composition of claim 1, wherein the cereal milling product is based on rye or spelt.

4. The composition of claim 1, wherein the cereal milling product is based on a cereal variety whose fraction of amylopectin is greater than that of amylose.

5. The composition of claim 1, wherein it contains the following:
approximately 29% by weight rye milling products,
approximately 12% by weight hemp seeds,
approximately 12% by weight soya meal,
approximately 3% by weight linseed,
approximately 2.5% by weight protein,
approximately 1.5% by weight sesame seed
approximately 1.5% by weight wheat bran
approximately 1.5% by weight milk protein, and also
approximately 1% by weight rice flakes.

6. The composition of claim 1, wherein it represents a baked good, in particular a bread.

7. A method for treating or preventing a disease in a human in need thereof, wherein the disease in is selected from the group consisting of cancer types which have an active TKTL-1 metabolism, diabetes, neurodegenerative diseases, in particular Alzheimer's disease, the method comprising the step of administering to the human a therapeutically effective amount of the composition as claimed in claim 1.
